(19) United States
(12) Patent Application Publication
(10) Pub. No.: US 2013/0171647 A1
(43) Pub. Date: Jul. 4, 2013

(54) PREDICTING RESPONSES TO ANDROGEN DEPRIVATION THERAPY

(71) Applicants: Douglas W. Mahoney, Elgin, MN (US); Manish Kohli, Rochester, MN (US); James R. Cerhan, Rochester, MN (US); Steven M. Offer, Rochester, MN (US)

(72) Inventors: Douglas W. Mahoney, Elgin, MN (US); Manish Kohli, Rochester, MN (US); James R. Cerhan, Rochester, MN (US); Steven M. Offer, Rochester, MN (US)

(73) Assignee: MAYO FOUNDATION FOR MEDICAL EDUCATION AND RESEARCH, Rochester, MN (US)

(21) Appl. No.: 13/821,807

(22) PCT Filed: Sep. 8, 2011
(86) PCT No.: PCT/US2011/050836

§ 371 Date: Mar. 8, 2013

Related U.S. Application Data
(60) Provisional application No. 61/380,786, filed on Sep. 8, 2010.

(51) Int. Cl. C12Q 1/68

(52) U.S. Cl. CPC C12Q 1/6886
USPC 435/6.11

(57) ABSTRACT

This document provides methods and materials for identifying prostate cancer patients likely to respond to an androgen deprivation therapy. For example, methods and materials for identifying a prostate cancer patient likely to respond to an androgen deprivation therapy based at least in part on the presence of a genetic variation in a TMRT11 nucleic acid are provided. This document also provides methods and materials for identifying prostate cancer patients likely to survive prostate cancer related death for a short or long period of time. For example, methods and materials for identifying a prostate cancer patient likely to survive prostate cancer related death for a short or long period of time based at least in part on the presence of a genetic variation in a UGT1A3 nucleic acid, a UGT1A7 nucleic acid, and/or a UGT1A10 nucleic acid are provided.

### CROSS-REFERENCE TO RELATED APPLICATIONS

This application claims priority to U.S. Provisional Application Ser. No. 61/380,786, filed on Sep. 8, 2010. The disclosure of the prior application is considered part of (and are incorporated by reference in) the disclosure of this application.

### BACKGROUND

1. Technical Field

This document relates to methods and materials involved in predicting whether a prostate cancer patient is likely to respond to an androgen deprivation therapy for a prolonged time period. For example, this document provides methods and materials for predicting whether a prostate cancer patient is likely to respond to an androgen deprivation therapy for a prolonged time period (e.g., greater than three years) based at least in part on the presence of a genetic variation in a TMRT11 nucleic acid.

This document also relates to methods and materials involved in determining whether a prostate cancer patient is likely to survive prostate cancer related death for a short or long period of time regardless of prostate cancer treatment (e.g., after failing androgen deprivation therapy). For example, this document provides methods and materials for determining whether a prostate cancer patient is likely to survive prostate cancer related death for a short or long period of time regardless of prostate cancer treatment after failing androgen deprivation therapy based at least in part on the presence of a genetic variation in a UGT1A3 nucleic acid, the presence of a genetic variation in a UGT1A7 nucleic acid, and/or the presence of a genetic variation in a UGT1A10 nucleic acid.

2. Background Information

Prostate cancer occurs when a malignant tumor forms in the tissue of the prostate. The prostate is a gland in the male reproductive system located below the bladder and in front of the rectum. The main function of the prostate gland, which is about the size of a walnut, is to make fluid for semen. Although there are several cell types in the prostate, nearly all prostate cancers start in the gland cells. This type of cancer is known as adenocarcinoma.

Prostate cancer is the second leading cause of cancer-related death in American men. Most of the time, prostate cancer grows slowly. Autopsy studies show that many older men who died of other diseases also had prostate cancer that neither they nor their doctor were aware of. Sometimes, however, prostate cancer can grow and spread quickly. When localized to the prostate, treatments are delivered with curative intent, either with surgical prostatectomy or radiation. Clinical follow up post treatment is performed by monitoring serum prostate specific antigen (PSA), which can become immeasurable after successful localized therapy. However, in a large case series with adequate longitudinal follow-up, between 27% and 53% of men undergoing radical prostatectomy were detected to have a PSA elevation (also labeled-biochemical failure) within 10 years following primary prostate therapy (surgery) signaling the first evidence of progressive disease prior to the appearance of clinical metastasis. An initial treatment after biochemical failure and progression to advanced disease can be continuous androgen deprivation therapy (ADT), which is usually performed in the United States by using intra-muscular or subcutaneous depots of luteinizing hormone-releasing hormone (LHRH)-analogues, every three to four months.

### SUMMARY

This document provides methods and materials for predicting whether a prostate cancer patient is likely to respond to an androgen deprivation therapy for a prolonged period of time. For example, this document provides methods and materials for predicting whether a prostate cancer patient is likely to respond to an androgen deprivation therapy for a prolonged period of time based at least in part on the presence of a genetic variation in a TMRT11 nucleic acid (e.g., rs6900796, rs1268121, rs2326215, and/or rs6569442). This document also provides methods and materials for predicting how long a prostate cancer patient is likely to respond to an androgen deprivation therapy based on the presence or absence of a genetic variation.

Having the ability to identify prostate cancer patients that are likely to respond to an androgen deprivation therapy can allow doctors and patients to proceed with appropriate treatment options. For example, a patient identified as having one or two alleles having rs6900796 or rs1268121 can be instructed to proceed with an ADT sooner than he would have been had he lacked alleles having rs6900796 or rs1268121.

This document also provides methods and materials for determining whether a prostate cancer patient is likely to survive prostate cancer related death for a short or long period of time regardless of prostate cancer treatment. For example, this document provides methods and materials for determining whether a prostate cancer patient is likely to survive prostate cancer related death for a short or long period of time regardless of prostate cancer treatment based at least in part on the presence of a genetic variation in a UGT1A3 nucleic acid (e.g., rs17864701, rs17862875, or rs11891311), the presence of a genetic variation in a UGT1A7 nucleic acid (e.g., rs6753320 or rs6736508), and/or the presence of a genetic variation in a UGT1A10 nucleic acid (e.g., rs10929251 or rs10929252).

As described herein, the presence of a genetic variation in a UGT1A3 nucleic acid (e.g., rs17864701, rs17862875, or rs11891311) can indicate that the cancer patient is likely to experience longer survival from prostate cancer regardless of the type of treatment as shown in FIGS. 8-10. The presence of a genetic variation in a UGT1A7 nucleic acid (e.g., rs6753320 or rs6736508) can indicate that the cancer patient is likely to experience longer survival from prostate cancer regardless of the type of treatment as shown in FIGS. 11 and 12. The presence of a genetic variation in a UGT1A10 nucleic acid (e.g., rs10929251 or rs10929252) can indicate that the cancer patient is likely to experience shorter survival from prostate cancer regardless of the type of treatment as shown in FIGS. 5 and 7.

Having the ability to identify prostate cancer patients that are likely to experience short survival time (e.g., less than three years) can allow doctors and patients to proceed with appropriate treatment options. For example, a patient identified as being likely to experience short survival time can be instructed to proceed with aggressive or additional treatment options including participation in clinical trials of new medications over and beyond the standard treatment options available, while a patient identified as being likely to experience long survival time can be instructed to proceed with standard treatment options alone.

In general, one aspect of this document features a method for identifying a prostate cancer patient likely to respond to androgen deprivation therapy. The method comprises, or consists essentially of, (a) detecting the presence of a TMRT11 allele comprising rs6900796 or rs1268121 in the patient, and (b) classifying the patient as being likely to respond to the androgen deprivation therapy without failure for a time greater than 3.5 years based at least in part on the presence of the TMRT11 allele. The prostate cancer patient can be a human. The method can comprise detecting the presence of a TMRT11 allele comprising rs6900796. The method can comprise detecting the presence of a TMRT11 allele comprising rs1268121.

In another aspect, this document features a method for identifying a prostate cancer patient likely to survive death related to prostate cancer for a time longer than 3.5 years. The method comprises, or consists essentially of, (a) detecting the presence of a UGT1A3 allele comprising rs17864701, rs17862875, or rs11891311 in the patient, and (b) classifying the patient as being likely to survive death related to prostate cancer for a time longer than 3.5 years based at least in part on the presence of the UGT1A3 allele. The prostate cancer patient can be a human. The method can comprise detecting the presence of a UGT1A3 allele comprising rs17864701. The method can comprise detecting the presence of a UGT1A3 allele comprising rs17862875. The method can comprise detecting the presence of a UGT1A3 allele comprising rs11891311.

In another aspect, this document features a method for identifying a prostate cancer patient likely to survive death related to prostate cancer for a time longer than 3.5 years. The method comprises, or consists essentially of, (a) detecting the presence of a UGT1A7 allele comprising rs6753320 or rs6736508 in the patient, and (b) classifying the patient as being likely to survive death related to prostate cancer a time longer than 3.5 years based at least in part on the presence of the UGT1A7 allele. The prostate cancer patient can be a human. The method can comprise detecting the presence of a UGT1A7 allele comprising rs6753320. The method can comprise detecting the presence of a UGT1A7 allele comprising rs6736508.

In another aspect, this document features a method for identifying a prostate cancer patient likely to survive death related to prostate cancer for a time longer than 3.5 years. The method comprises, or consists essentially of, (a) detecting the presence of two UGT1A10 alleles comprising a wild-type sequence at the rs10929251 or rs10929252 SNP position in the patient, and (b) classifying the patient as being likely to survive death related to prostate cancer a time longer than 3.5 years based at least in part on the presence of the two UGT1A10 alleles. The prostate cancer patient can be a human. The method can comprise detecting the presence of two UGT1A10 alleles comprising a wild-type sequence at the rs10929251 SNP position. The method can comprise detecting the presence of two UGT1A10 alleles comprising a wild-type sequence at the rs10929252 SNP position.

In another aspect, this document features a method for identifying a prostate cancer patient likely to survive death related to prostate cancer for a time shorter than 3.0 years. The method comprises, or consists essentially of, (a) detecting the presence of a UGT1A10 allele comprising rs10929251 or rs10929252 in the patient, and (b) classifying the patient as being likely to survive death related to prostate cancer for a time shorter than 3.0 years based at least in part on the presence of the UGT1A10 allele. The prostate cancer patient can be a human. The method can comprise detecting the presence of a UGT1A10 allele comprising rs10929251. The method can comprise detecting the presence of a UGT1A10 allele comprising rs10929252.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is map of biosynthetic pathways. Gene names are in bold italics. Abbreviations: HSDs, hydroxysteroid dehydrogenases; 3β-HSD2, 3β-hydroxysteroid dehydrogenase type 2.

FIG. 2 is graph plotting the time to ADT failure (year) for prostate cancer patients with zero, one, or two TRMT11 alleles having rs6900796. The arms represent the distribution of patient survival periods for the whole group (Y-axis-time to failure after initiating androgen deprivation therapy); the box represents the interquartile range (IQ25-IQ75); and the thick black bar represents median time periods. HR stands for hazard ratio for the highly significant p-value shown. A HR of 0.74 means that there is a greater than 25% likelihood of patients with no alleles for this SNP to have a shorter survival while receiving androgen deprivation therapy for cancer—median of 2.53 years compared to a median of 3.84 years.

FIG. 3 is graph plotting the time to ADT failure (year) for prostate cancer patients with zero, one, or two TRMT11 alleles having rs1268121. The arms represent the distribution of patient survival periods for the whole group (Y-axis-time to failure after initiating androgen deprivation therapy); the box represents the interquartile range (IQ25-IQ75); and the thick black bar represents median time periods. HR stands for hazard ratio for the highly significant p-value shown. A HR of 0.66 means that there is a greater than 33% likelihood of patients with no alleles for this SNP to have a shorter survival while receiving androgen deprivation therapy for cancer—median of 3.08 years compared to a median of 5.86 years.

FIG. 4 is graph plotting the time to ADT failure (year) for prostate cancer patients with zero, one, or two TRMT11 alleles having rs1268121 and/or rs6900796. The arms represent the distribution of patient survival periods for the whole group (Y-axis-time to failure after initiating androgen deprivation therapy); the box represents the interquartile range (IQ25-IQ75); and the thick black bar represents median time periods. This figure is a multivariate analyses. HR stands for hazard ratio for the highly significant p-value shown. A HR of 0.81 for rs6900796 means that there is a greater than 19% likelihood of patients with no alleles for this SNP to have a shorter survival while receiving androgen deprivation therapy for cancer independent of the effects on survival of the other SNP (rs1268121).

FIG. 5 is graph plotting the percent mortality versus time (years) for prostate cancer patients with zero, one, or two UGT1A10 alleles having rs10929251.

FIG. 6 is graph plotting the percent mortality versus time (years) for prostate cancer patients with zero, one, or two UGT1A10 alleles having rs1823803.

FIG. 7 is graph plotting the percent mortality versus time (years) for prostate cancer patients with zero, one, or two UGT1A10 alleles having rs10929252.

FIG. 8 is graph plotting the percent mortality versus time (years) for prostate cancer patients with zero, one, or two UGT1A3 alleles having rs17864701.

FIG. 9 is graph plotting the percent mortality versus time (years) for prostate cancer patients with zero, one, or two UGT1A3 alleles having rs17862875.

FIG. 10 is graph plotting the percent mortality versus time (years) for prostate cancer patients with zero, one, or two UGT1A3 alleles having rs11891311.

FIG. 11 is graph plotting the percent mortality versus time (years) for prostate cancer patients with zero, one, or two UGT1A7 alleles having rs6753320.

FIG. 12 is graph plotting the percent mortality versus time (years) for prostate cancer patients with zero, one, or two UGT1A7 alleles having rs6736508.

FIG. 13 is graph plotting the percent mortality versus time (years) for prostate cancer patients with zero, one, or two UGT1A7 alleles having rs17864689.

### DETAILED DESCRIPTION

This document provides methods and materials for predicting whether or not a prostate cancer patient is likely to respond to an androgen deprivation therapy based at least in part on the presence of a genetic variation in a TMRT11 nucleic acid (e.g., rs6900796, rs1268121, rs2326215, or rs6569442). As described herein, a mammal (e.g., a human) that contains one or two TMRT11 alleles of rs6900796 or rs1268121 can be identified as being likely to experience prolonged response to an ADT (e.g., likely to experience greater than 2.5 years of survival prior to ADT failure). For example, a prostate cancer patient having one or two TMRT11 alleles of rs6900796 or rs1268121 can be classified as being likely to experience greater than 2.5 years (e.g., greater than 2.5, 2.6, 2.7, 2.8, 2.9, 3.0, 3.5, 4.0, 4.5, 5.0, or 5.5 years) of survival without ADT failure. Examples of ADT include, without limitation, chemical castrations (e.g., treatments with LHRH-analogues or gonadotrophin-releasing hormone (GnRH) antagonists) and physical castrations.

This document also provides methods and materials for determining whether a prostate cancer patient is likely to survive prostate cancer related death for a short or long period of time regardless of prostate cancer treatment based at least in part on the presence of a genetic variation in a UGT1A3 nucleic acid (e.g., rs17864701, rs17862875, or rs11891311), the presence of a genetic variation in a UGT1A7 nucleic acid (e.g., rs6753320 or rs6736508), and/or the presence of a genetic variation in a UGT1A10 nucleic acid (e.g., rs10929251 or rs10929252). As described herein, a mammal (e.g., a human) that contains one or two UGT1A3 alleles of rs17864701, rs17862875, or rs11891311 and/or one or two UGT1A7 alleles of rs6753320 or rs6736508 can be identified as being likely to experience survival from prostate cancer longer than 2.5 years (e.g., longer than 2.5, 2.6, 2.7, 2.8, 2.9, 3.0, 3.5, 4.0, 4.5, 5.0, or 5.5 years) regardless of the type of treatment. A mammal (e.g., a human) that contains two wild-type UGT1A10 alleles at the position of the rs10929251 and/or rs10929252 SNPs can be identified as being likely to experience survival from prostate cancer longer than 2.5 years (e.g., longer than 2.5, 2.6, 2.7, 2.8, 2.9, 3.0, 3.5, 4.0, 4.5, 5.0, or 5.5 years) regardless of the type of treatment. In some cases, a mammal (e.g., a human) that contains one or two UGT1A10 alleles of rs10929251 and/or rs10929252 can be identified as being likely to experience survival from prostate cancer shorter than 2.5 years regardless of the type of currently available treatment.

Any appropriate method can be used to detect the presence of one or two alleles of a particular SNP provided herein. For example, mutations can be detected by sequencing cDNA, untranslated sequences, denaturing high performance liquid chromatography (DHPLC; Underhill et al., Genome Res., 7:996-1005 (1997)), allele-specific hybridization (Stoneking et al., Am. J. Hum. Genet., 48:370-382 (1991); and Prince et al., Genome Res., 11(1):152-162 (2001)), allele-specific restriction digests, mutation specific polymerase chain reactions, single-stranded conformational polymorphism detection (Schafer et al., Nat. Biotechnol., 15:33-39 (1998)), infrared matrix-assisted laser desorption/ionization mass spectrometry (WO 99/57318), and combinations of such methods.

In some cases, genomic DNA can be used to detect an allele having a SNP provided herein. Genomic DNA typically is extracted from a biological sample such as a peripheral blood sample, but can be extracted from other biological samples, including tissues (e.g., mucosal scrapings of the lining of the mouth or from prostate tissue). Any appropriate method can be used to extract genomic DNA from a blood or tissue sample, including, for example, phenol extraction. In some cases, genomic DNA can be extracted with kits such as the QIAamp® Tissue Kit (Qiagen, Chatsworth, Calif.), the Wizard® Genomic DNA purification kit (Promega, Madison, Wis.), the Puregene DNA Isolation System (Gentra Systems, Minneapolis, Minn.), or the A.S.A.P.3 Genomic DNA isolation kit (Boehringer Mannheim, Indianapolis, Ind.).

An amplification step can be performed before proceeding with the detection method. For example, TMRT11, UGT1A3, UGT1A7, and/or UGT1A10 nucleic acid can be amplified and then directly sequenced. Dye primer sequencing can be used to increase the accuracy of detecting heterozygous samples.

This document also provides methods and materials to assist medical or research professionals in determining whether or not a prostate cancer patient is likely to respond to an androgen deprivation therapy and methods and materials to assist medical or research professionals in determining whether a prostate cancer patient is likely to survive prostate cancer related death for a short or long period of time regardless of prostate cancer treatment. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the presence of one or more alleles having a SNP described herein, and (2) communicating information about that SNP to that professional.

Any method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

In some cases, a patient identified as being likely to respond to an androgen deprivation therapy based at least in part on the presence of a genetic variation in a TMRT11 nucleic acid (e.g., rs6900796, rs1268121, rs2326215, or rs6569442) can be administered or instructed to receive an alternative or adjunct therapy to ADT. For example, a patient having one or two alleles having rs6900796 or rs1268121 can be instructed to proceed with an ADT in conjunction with an additional treatment such as abiraterone acetate, MDV3100, or TAK-700 sooner than he would have been had he lacked alleles having rs6900796 or rs1268121. In some cases, a patient identified as being likely to respond to an androgen deprivation therapy based at least in part on the presence of a genetic variation in a TMRT11 nucleic acid (e.g., rs6900796, rs1268121, rs2326215, or rs6569442) can be administered or instructed to receive abiraterone acetate, MDV3100, TAK-700, or a combination thereof.

The invention will be further described in the following examples, which do not limit the scope of the invention described in the claims.

### EXAMPLES

### Example 1

### Identifying Genotypic Markers Associated with ADT Response

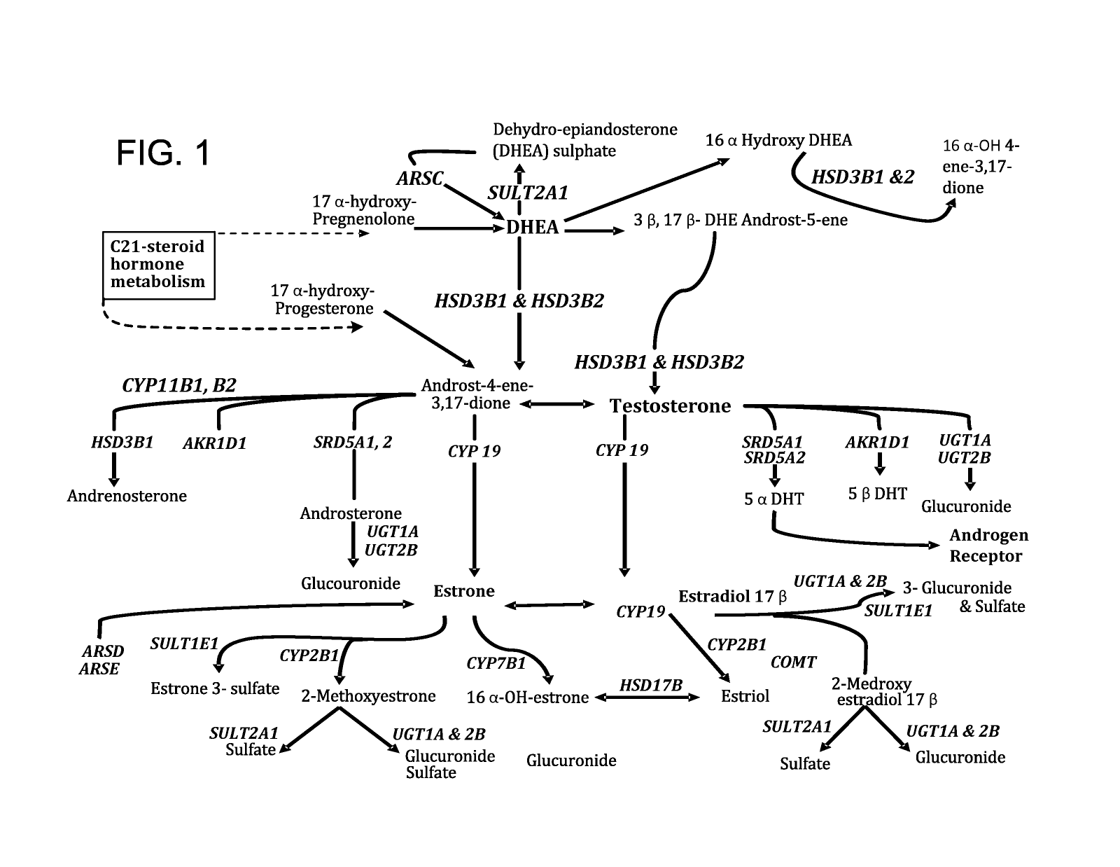

A candidate gene/SNP based association line of investigation into the variation in genes regulating hormonal pathways in a homogenous population of prostate cancer subjects receiving ADT was performed with the overall goal of identifying specific genetic markers associated with ADT response. The study included tagSNPs in candidate genes known to be involved in sex steroid synthesis and metabolism and included definitive survival endpoints for associating response or failure of ADT. The candidate genes can be divided into four biosynthetic pathways (FIG. 1):

i) C4 Δ pathway (nucleic acids that encode enzymes that convert progesterone to androstenedione);

ii) C5 Δ pathway (nucleic acids that encode enzymes that convert cholesterol to pregnenolone to dihydro-epiandosterone);

iii) C21 CYP pathway (CYP17 17α-hydroxylates all four classes of 21-carbon steroids, and the 17,20-lyase activity for each pathway can vary); and

iv) “Backdoor pathway” for androgen synthesis (nucleic acids that encode enzymes involved in the conversion of 17-hydroxyprogesterone to 5alpha-reduced androgen precursors via a 5alpha-reductase type 1 enzyme).

The metabolism pathway nucleic acids for androgens included SRD5A1, SRD5A2, CYP19, UGT1A, UGT2B, AKR1D1, SULT1E1, CYP2B1, COMT, CYP7B1, HSD17B, SULT2A1, ARSD, ARSE, and TRMT11. The end products of several of these pathways were 2-methoxyestrone, estriol, sufate and glucouronides, estrone-3-sulphate.

For determining genomic classifiers as predictive factors for ADT responses, three different patient data sets were used:

i) A cohort of prostate cancer patients available at Mayo Clinic Clinical Core treated with ADT on whom long term clinical outcomes of treatment and follow up is available (n=258).

ii) Specimens from patients from the University of Rochester, N.Y.

iii) DNA specimens from advanced prostate cancer patients receiving or who have received androgen ablation collected at the Mayo clinic (n=42).

A total of 338 patients were identified in the above three databases that met the criteria of being treated with hormone therapy (also referred to as androgen ablation or androgen deprivation therapy or ADT). Demographic and disease characteristics of the 338 patient cohort were summarized (Table 1).

TABLE 1

| Clinical Characteristic | N (%) | |
|---|---|---|
| Race | | |
| Caucasian (%) | 301 | (99%) |
| Others | 3 | (1%) |
| Age (years) (N = 304) | | |
| Median (Q1-Q3) | 72 | (47-91) |
| PSA at the time of ADT failure (ng/ml) | | |
| Median (range) | 11.5 | (4-46) |
| Clinical T stage at first diagnosis | | |
| T1 | 18 | (6) |
| T2 | 127 | (42) |
| T3 + 4 | 16 | (5) |
| T4 | 41 | (13) |
| TX (un-verifiable) | 102 | (34) |
| N0 | 37 | (13) |
| N1 | 4 | (2) |
| NX | 263 | (87) |
| Patients with no metastatic disease at diagnosis-M0 | 279 | (92) |
| Patients with metastatic disease at diagnosis-M1 | 23 | (7) |
| Mx (unverifiable) | 2 | (1) |
| Time from diagnosis to ADT initiation (years) | | |
| For initially non-metastatic patients (MO): Median | 1.5 | |
| (Q1, Q3) | (19 days-5 years) | |
| For initially metastatic patients (Ml): Median (Q1, Q3) | 16 days | |
| | (0 days-31 days) | |
| Biopsy Gleason score at initial diagnosis | | |
| <6 | 65 | (21) |
| 7 | 90 | (30) |
| >8 | 121 | (40) |
| Unknown | 28 | (9) |
| Definitive local therapy | | |
| None | 85 | (28) |
| Radical prostatectomy | 131 | (43) |
| Radiation Therapy | 88 | (29) |
| Type of ADT | | |
| LHRH analogue | 210 | (69) |
| Orchiectomy | 94 | (31) |

Genotypes among nearby common genetic polymorphisms tend to be correlated. Selecting and prioritizing representative ‘tag’ SNPs improved the cost-effectiveness of the genetic study. The genetic structure of 84 candidate genes, including a subset of 57 candidate genes, involved in testosterone metabolism were evaluated by interrogating publicly available genotype data for European populations from the International HapMap Phase II (http://www.hapmap.org), Seattle SNPs (http://pga.mbt.washington.edu/), and NIEHS SNPs (http://egp.gs.washington.edu/) projects. SNPs spanning 5 kilobases upstream and downstream of each gene were used for genetic characterization in the 60 unrelated HapMap CEU samples (chromosomal position of genes and SNPs were extracted from RefSeq release 29, NCBI build 36, and dbSNP build 129). SNPs from Hapmap were found in 78 of the candidate genes. Six of the candidate genes were each resequenced in NIEHS SNPs and Seattle SNPs. Two of the genes had no SNPs. To thoroughly capture the common genetic variation, a pairwise tagging approach was utilized on each gene and each genotype source separately such that all SNPs with reported and pre-determined minor allele frequency (MAF)>=5% were either directly measured or were highly correlated (R²>=0.9) with a measured SNP.

For genes with more than one genotype source, an optimal source of tagSNPs was selected based on the one with more LD bins, giving priority to HapMap in case of equal number of bins. Hapmap was selected as an optimal source for 74 of the genes, Seattle for three of the genes, and NIEHS for one of the genes. For the subset of 57 candidate genes, the HapMap was chosen as the best source for 57 genes and NIEHS for 1 gene. To pick an optimal tagSNP for each LD bin, the SNPPicker software developed in the Mayo Clinic Bioinformatics group was used. LdSelect often gives multiple choices of tagSNPs for a given bin but not all tagSNPs have the same design probability or possible functional relevance. SNPPicker picks an optimal tagSNP for each bin, optimizing constraints such as assay score, functional relevance, and the Illumina GoldenGate platform constraint of not allowing two SNPs that are 60 bp or less from each other. It also allows multiple tagSNPs for bins. To reduce the probability of failure in larger bins, three tagSNPs were selected for bins with >=30 SNPs, while two were selected for bins with size of 10 or greater. All tagSNPs met the minimum Illumina assay score of 0.4. To increase the likelihood of identifying susceptibility alleles, 149 SNPs of interest from various sources (likely to be functionally deleterious, previous experiment evidence of association, etc.) were chosen in preference to other tagSNPs or added to the final list for a total of 1056 SNPs.

A total of 755 tagSNPs in the 58 candidate genes were selected for genotyping. In addition, 69 targeted candidate SNPs were genotyped from 20 candidate genes also in sex steroid biosynthesis and metabolism genes of which 6 were already included in the tagged set, but none of the SNPs overlapped. These candidate SNPs were selected base on previous published data from single patient cohorts suggesting either a potential functionality for the SNP or a potential significant association with response to ADT.

### SNPPicker

There are several popular programs (e.g., ldselect, tagzilla, and tagger) that help a user save on genotyping costs by selecting sets of highly correlated SNPs (called bins) and only genotyping one (or a few) representative tag SNP. A tagSNP is selected if it correlates well with all the other SNPs in the bin above some correlation coefficient (r²) threshold. The output of these programs often gives multiple choices of tag SNPs for a given bin. However, not all tag SNPs have the same design probability or even the same functional importance.

SNPPicker is a post-processor to these bin-based algorithms that can refine the list of tag SNPs subject to multiple realistic constraints, including the 60 base pair constraints for Illumina. Using a three step algorithm, SNPPicker rejects solutions incompatible with the constraint, rapidly finds a good solution, and then spends as much time as the user allows looking for an optimal solution. SNPPicker is able to split SNPs that are too close among multiple SNP panels (user option) and can deal with multi-population SNP selection as well as cases where the bins are from multiple overlapping sources (e.g. Hapmap and Seatle SNPs) for the same population. SNPPicker depends on input files providing some information about the SNPs. The default format is designed to work with the Illumina provided annotation files that a user can get from Illumina.

Using supplied annotation and historical data on SNP assay performance, SNPPicker starts by computing the probability of successfully designing each SNP. Using that probability, it computes the utility of the panel, namely the sum of the probabilities of successfully genotyping each bin times the number of SNPs in that bin divided by the number of tag SNPs in the panel.

To allow flexibility in optimizing functionally relevant SNPs, the probability for all the SNPs are assessed in fixed intervals, so that within a given probability interval, priority is given to SNPs with stronger functional consequence. The functional ranking is configurable, but the default rank mapping uses the annotation in Illumina files and functional ranking of ldselect. As defined, the utility function considers bins that share SNPs, so that SNPs that tag more than one bin (e.g., in multi-population tagging or with overlapping bins from neighboring regions) improve the utility of the panel (subject to the probability). The utility function favors large bins over singletons and also allows multiple tag-SNPs to be selected for one bin in order to improve the probability that these larger bins will not fail.

The first step in SNPPicker is to filter out SNPs below a certain score cutoff. The remaining SNPs are chosen according to the following scoring: Proximity constraints must be met (though tag SNPs and obligates can be split across multiple panels), then the utility function is optimized. Given two solutions with the same utility (or choice between two SNPs with same probability), the next consideration is the functional importance of a SNP, the last consideration is the score of a SNP (since two SNPs with the same probability can have different scores).

### Genotyping the Custom “SNP-Chip”

Germline DNA purified from the above specimens were used for Illumina GoldenGate assay (GGGT) with the 936 SNPs selected, including the 824 SNPs selected subset (755 tagSNPs belonging to 58 selected candidate genes plus 69 selected tagSNPs). Illumina GGGT assays used well established protocols for performing the genotyping, which typically encompass primer extension, ligation, and universal PCR in very highly-plexed reactions (384-1536 plex). For GGGT, SNPs and genes were submitted for assay design. Location within current build of the genome was required for all submissions, and a RefSeq number identified genes. Primers were designed for each multiplex panel, and each SNP was rated for its probability of yielding optimal results for the GGGT biochemistry, on a scale of 0-1.

Analyses of the Genotypes Generated on 338 Advanced Prostate Cancer Patients Treated with Androgen Ablation

Genotypes were generated on samples from 338 prostate cancer and three CEPH subjects for 936 SNPs, including the 824 SNPs selected subset. For quality assurance, eight of the 338 prostate cancer samples were duplicated twice within the same plates, while the CEPH samples were genotyped multiple times within and across plates. All pairwise replicate sample comparisons exhibited a 100% genotype call concordance rate. Duplicated samples with a lower call rate together with the CEPH samples were eliminated from the subsequent statistical analysis. Of the remaining 338 samples, eight generated no genotypes and were therefore excluded. Evaluation of paired identity by state revealed five related pairs of samples. These paired samples were independently confirmed to have came from different blood draws of the same subjects. Only the sample with the higher call rate was retained for each of the five subjects.

25 SNPs (15 SNPs from the 824 SNPs of the selected subset) were omitted due to failed assays (0% call rate). Since all the X-linked genes of this study lie outside of the pseudo-autosomal regions, males can only have homozygous genotype for the relevant SNPs. However, five X-linked loci were identified with at least one heterozygote male: 3 SNPs has one, 1 SNP has two, and the other has 127 heterozygote males (of the 824 SNPs of the selected subset, 1 X-linked SNP was identified with excessive heterozygosity). The corresponding genotypes of the first four SNPs were set to missing, and the fifth SNP was discarded. Sixty SNPs (48 SNPs from the 824 SNPs of the selected subset) were eliminated because of low MAF (<5% in this study population where only females were included in the calculation of the X-link SNPs). 16 SNPs were dropped based on a stringent call rate of 98%. Five SNPs (two SNPs from the 824 SNPs of the selected subset) deviated from the Hardy-Weinberg equilibrium (Chi-square p-value<0.0001; only females contributed to the calculation of the X-linked SNPs). Upon visual inspection of the genotype clusters, two out of the five SNPs were omitted due to poor clustering quality. After discarding two prostate cancer samples with call rates less than 98%, a final dataset of 323 samples and 747 SNPs or 746 SNPs was used for further analysis.

Clinical data was incomplete for another 19 patients. A final total of 304 patients were identified for the statistical analyses with response to ADT (hormonal therapy).

### Results

Of the 84 hormone metabolizing genes analyzed for association with duration of response to ADT, variation in TRMT11 (tRNA methyltransferase 11 homologue; synonyms: TRM11, MDS024, C6orf75, and TRMT11-1) was strongly associated with ADT response (p<0.0008; adjusted p-value for FDR-0.068). TRMT11 nucleic acid encodes a polypeptide that is implicated in breaking down testosterone (indirectly) and estrogen (directly) into sulphone and glucouronide by-products. When evaluating the TRMT11 nucleic acid in the patient population without adjustments for age and Gleason score as discussed, the p-value for the TRMT11 gene was 0.001264 with and FDR=0.014045.

At the gene-level analysis, statistical significance (P<0.05) was observed for three genes (TRMT11, HSD17B12, and PRMT3) with time to ADT failure after adjusting for Gleason Score (Table 2), with a suggestive trend (p-value ≦0.07, and a corresponding FDR of 0.80) for an additional gene, WBSCR22—listed in Table 3). Of these, TRMT11 (tRNA methyltransferase 11 homologue) was the most significant gene (p=0.1×10⁻³; FDR=0.008).

TABLE 2

| | | | Number of | | Principal | |
|---|---|---|---|---|---|---|
| | | | tagSNPs | | Components | |
| | | | modeled via | Principal | Analysis | False |
| | Number | Number of | Principal | Components | Gleason | Discovery |
| GENE | of | tagSNPs with | Component | Analysis P- | Adjusted P- | Rate |
| Symbol | tagSNPs | p-value < 0.05 | Analysis | value | value | (q-value) |
| TRMT11 | 4 | 2 | 2 | 0.1 × 10⁻³ | 0.008 | 0.008 |
| HSD17B12 | 30 | 2 | 9 | 0.02 | 0.25 | 0.47 |
| PRMT3 | 23 | 2 | 5 | 0.05 | 0.06 | 0.81 |

TABLE 3

| | | | Number of SNPs | |
|---|---|---|---|---|
| | | | successfully | |
| GENE | P-Value | FDR | genotyped | Tagging SNPs |
| TRMT11 | 0.0001 | 0.0083 | 4 | rs2326215 rs6569442 rs6900796 |
| | | | | rs1268121 |
| HSD17B12 | 0.016 | 0.4654 | 30 | rs7110437 rs10838157 rs1878764 |
| | | | | rs12277307 rs4359199 rs10838159 |
| | | | | rs2037296 rs10838175 rs7120203 |
| | | | | rs4755737 rs11037556 rs7928523 |
| | | | | rs6485453 rs938942 rs7928182 |
| | | | | rs12800235 rs11037685 rs9783388 |
| | | | | rs9783294 rs1061810 rs10838172 |
| | | | | rs11037567 rs17597526 rs11037662 |
| | | | | rs10768989 rs11037634 rs11037589 |
| | | | | rs1518820 rs7110523 rs16937624 |
| PRMT3 | 0.0544 | 0.805 | 23 | rs6483694 rs736283 rs16906280 |
| | | | | rs10734292 rs3758803 rs11025567 |
| | | | | rs11025588 rs7114918 rs7115830 |
| | | | | rs10833325 rs17232653 rs10833332 |
| | | | | rs7396037 rs7941826 rs11025592 |
| | | | | rs1899483 rs11025571 rs10833341 |
| | | | | rs11025559 rs10833328 rs10741842 |
| | | | | rs12420525 rs2403587 |
| WBSCR22 | 0.0648 | 0.805 | 2 | rs2293488 rs1001220 |
| CYP3A4 | 0.0921 | 0.805 | 4 | rs12333983 rs2242480 rs4646437 |
| | | | | rs11773597 |
| PRMT2 | 0.0935 | 0.805 | 11 | rs6518306 rs2256070 rs2839376 |
| | | | | rs2839377 rs2839378 rs4819271 |
| | | | | rs7116 rs2236617 rs6518305 |
| | | | | rs7283192 rs7510435 |
| SULT2B1 | 0.0972 | 0.805 | 27 | rs2544785 rs10419482 rs3760802 |
| | | | | rs8108904 rs12462337 rs6509396 |
| | | | | rs1236093 rs10417472 rs2665581 |
| | | | | rs1132054 rs3826827 rs2544796 |
| | | | | rs3760808 rs12460535 rs4149455 |
| | | | | rs3848542 rs10426628 rs2302948 |
| | | | | rs10426377 rs279451 rs12611137 |
| | | | | rs2665605 rs3760804 rs2665579 |
| | | | | rs2544795 rs2665582 rs2665601 |
| SRD5A1 | 0.1348 | 0.977 | 14 | rs248799 rs482121 rs3822430 |
| | | | | rs7720479 rs248805 rs471604 |
| | | | | rs1560149 rs494958 rs3797177 |
| | | | | rs39848 rs30434 rs39847 rs518673 |
| | | | | rs16877779 |
| AKR1D1 | 0.1681 | 0.9825 | 13 | rs10954602 rs2166188 rs1872929 |
| | | | | rs12111721 rs2306846 rs6467736 |
| | | | | rs1817686 rs17169507 rs2035648 |
| | | | | rs7785788 rs2633359 rs3735023 |
| | | | | rs6467735 |
| UGT2A1 | 0.2024 | 0.9825 | 31 | rs17618178 rs7665571 rs10001991 |
| | | | | rs10903210 rs17147542 rs1158439 |
| | | | | rs1560605 rs4401516 rs4148309 |
| | | | | rs9992698 rs3775782 rs4148312 |
| | | | | rs1432315 rs6848997 rs17147521 |
| | | | | rs4280808 rs1432336 rs7656541 |
| | | | | rs10033854 rs7668703 rs11729544 |
| | | | | rs1347047 rs13134357 rs2163659 |
| | | | | rs7670819 rs1432313 rs1432329 |
| | | | | rs10518065 rs11249454 rs4148301 |
| | | | | rs10026988 |
| SULT1E1 | 0.212 | 0.9825 | 10 | rs12499679 rs10019305 rs1220702 |
| | | | | rs1220725 rs11573763 rs1220703 |
| | | | | rs1881668 rs4149535 rs4149525 |
| | | | | rs3775775 |
| HSD3B1 | 0.2193 | 0.9825 | 3 | rs1812256 rs6428830 rs6203 |
| UGT2A3 | 0.2292 | 0.9825 | 7 | rs2168841 rs2331562 rs2331563 |
| | | | | rs2168840 rs17147016 rs7679122 |
| | | | | rs3749514 |
| UGT2B11 | 0.2372 | 0.9825 | 3 | rs13123057 rs4400059 rs12502502 |
| UGT2B28 | 0.2821 | 0.9966 | 1 | rs7437560 |
| CYP19A1 | 0.2889 | 0.9966 | 44 | rs1143704 rs700518 rs4545755 |
| | | | | rs1902586 rs727479 rs2470176 |
| | | | | rs1961177 rs2470157 rs17601241 |
| | | | | rs1004984 rs2470146 rs1902585 |
| | | | | rs10851498 rs2445765 rs8025191 |
| | | | | rs2255192 rs16964258 rs10046 |
| | | | | rs12900487 rs17703883 rs12907866 |
| | | | | rs17601876 rs2470152 rs2470144 |
| | | | | rs11856927 rs10459592 rs12911554 |
| | | | | rs2899470 rs4441215 rs7172156 |
| | | | | rs3751592 rs2445762 rs2899472 |
| | | | | rs6493496 rs2414099 rs2008691 |
| | | | | rs3751591 rs7174997 rs8025374 |
| | | | | rs12439137 rs17523880 rs1902584 |
| | | | | rs10519295 rs17523922 |
| PRMT7 | 0.2975 | 0.9966 | 12 | rs9889191 rs1111571 rs3785114 |
| | | | | rs3785116 rs2863978 rs9934232 |
| | | | | rs4381598 rs10775303 rs2307022 |
| | | | | rs1530644 rs7197653 rs12599876 |
| METTL2B | 0.3373 | 0.9966 | 7 | rs2562737 rs1065267 rs1053124 |
| | | | | rs7779945 rs7779455 rs12530672 |
| | | | | rs4731458 |
| HSD17B3 | 0.3633 | 0.9966 | 28 | rs407179 rs867807 rs1887774 |
| | | | | rs379734 rs7026934 rs1324196 |
| | | | | rs999269 rs7029101 rs7037932 |
| | | | | rs11788785 rs2476923 rs7848739 |
| | | | | rs11788083 rs8190581 rs10820299 |
| | | | | rs6479179 rs8190512 rs10739847 |
| | | | | rs2257157 rs1927882 rs2026001 |
| | | | | rs1119864 rs394243 rs8190534 |
| | | | | rs2253502 rs8190536 rs9409407 |
| | | | | rs2479828 |
| LCMT1 | 0.4112 | 0.9966 | 13 | rs277894 rs277914 rs277891 |
| | | | | rs13338420 rs13334427 rs277898 |
| | | | | rs11642659 rs11645986 rs11860180 |
| | | | | rs13337201 rs11649654 rs3809680 |
| | | | | rs9630611 |
| UGT2B7 | 0.4129 | 0.9966 | 6 | rs7662029 rs4356975 rs6600894 |
| | | | | rs7435335 rs3924194 rs10028494 |
| SRD5A2 | 0.4147 | 0.9966 | 10 | rs632148 rs2268797 rs2281546 |
| | | | | rs6543634 rs2300701 rs12470143 |
| | | | | rs11690596 rs559555 rs7562326 |
| | | | | rs3754838 |
| CYP11B2 | 0.4552 | 0.9966 | 5 | rs1799998 rs7844961 rs6987382 |
| | | | | rs11781082 rs3097 |
| CARM1 | 0.4647 | 0.9966 | 6 | rs1549926 rs892011 rs12460421 |
| | | | | rs1529711 rs7254708 rs11670365 |
| METTL6 | 0.4821 | 0.9966 | 5 | rs13081119 rs13323290 rs13075694 |
| | | | | rs2290535 rs2290536 |
| HSD17B1 | 0.5123 | 0.9966 | 4 | rs2830 rs676387 rs12602084 |
| | | | | rs2676530 |
| HEMK1 | 0.5605 | 0.9966 | 2 | rs17787569 rs388483 |
| CYP11B1 | 0.5725 | 0.9966 | 7 | rs1134095 rs1134096 rs5301 |
| | | | | rs7833415 rs6410 rs4464947 rs5297 |
| ESR1 | 0.5728 | 0.9966 | 80 | rs9479193 rs3020411 rs3020422 |
| | | | | rs1884054 rs3020318 rs12665044 |
| | | | | rs3853248 rs2234693 rs9479130 |
| | | | | rs985694 rs2347867 rs1801132 |
| | | | | rs712221 rs12154178 rs6557170 |
| | | | | rs926779 rs722208 rs6557177 |
| | | | | rs3003921 rs1884052 rs827423 |
| | | | | rs2982896 rs3020394 rs3020403 |
| | | | | rs7761846 rs1709182 rs9340835 |
| | | | | rs12199722 rs2982683 rs3020314 |
| | | | | rs3020434 rs1709183 rs7757956 |
| | | | | rs6914211 rs9341052 rs11155813 |
| | | | | rs9340978 rs3020393 rs13203975 |
| | | | | rs12199102 rs9341016 rs3798577 |
| | | | | rs2982712 rs1543403 rs7755185 |
| | | | | rs2474148 rs3020376 rs532010 |
| | | | | rs488133 rs1884051 rs3020407 |
| | | | | rs2813543 rs2747649 rs9322335 |
| | | | | rs2813544 rs12212176 rs7743290 |
| | | | | rs3020328 rs9322336 rs926777 |
| | | | | rs985191 rs7775047 rs3003925 |
| | | | | rs988328 rs2228480 rs9397463 |
| | | | | rs2273206 rs3003917 rs3020368 |
| | | | | rs3020317 rs3020410 rs2144025 |
| | | | | rs3020383 rs9340788 rs2982894 |
| | | | | rs2982900 rs3778099 rs1062577 |
| | | | | rs9341066 rs9340789 |
| UGT2B10 | 0.5874 | 0.9966 | 3 | rs861340 rs11737566 rs4694358 |
| SERPINE1 | 0.6243 | 0.9966 | 8 | rs11178 rs2227672 rs2227631 |
| | | | | rs1050813 rs1050955 rs2227667 |
| | | | | rs4727479 rs2227692 |
| PRMT6 | 0.6378 | 0.9966 | 3 | rs1623927 rs3791185 rs2232016 |
| HSD11B1 | 0.6513 | 0.9966 | 14 | rs3766619 rs9430012 rs2205985 |
| | | | | rs11119328 rs4844880 rs2235543 |
| | | | | rs17389016 rs11808690 rs6672256 |
| | | | | rs846906 rs3753519 rs4844488 |
| | | | | rs846910 rs12565406 |
| THBS1 | 0.66 | 0.9966 | 4 | rs11070220 rs1051442 rs1478604 |
| | | | | rs2228262 |
| SULT2A1 | 0.6754 | 0.9966 | 8 | rs212099 rs2547229 rs7508610 |
| | | | | rs188440 rs182420 rs2547238 |
| | | | | rs2932766 rs2910393 |
| UGT2B4 | 0.7111 | 0.9966 | 13 | rs10518061 rs1845556 rs1826690 |
| | | | | rs11249442 rs1569343 rs941389 |
| | | | | rs7441743 rs1131878 rs2013573 |
| | | | | rs17614939 rs17671289 |
| | | | | seq_rs1389930 rs3822179 |
| PRMT5 | 0.7147 | 0.9966 | 4 | rs11157930 rs12589539 rs4981449 |
| | | | | rs8007089 |
| PRMT8 | 0.7162 | 0.9966 | 52 | rs12423361 rs7972007 rs1860450 |
| | | | | rs4766137 rs10491968 rs876594 |
| | | | | rs10848876 rs7137875 rs4766141 |
| | | | | rs10774158 rs6489480 rs11062733 |
| | | | | rs917602 rs6489474 rs1029766 |
| | | | | rs2159404 rs887303 rs7972248 |
| | | | | rs4765741 rs758637 rs11062731 |
| | | | | rs3782753 rs3759362 rs11062713 |
| | | | | rs10774156 rs3782744 rs7976970 |
| | | | | rs7966000 rs17696856 rs4766130 |
| | | | | rs11062694 rs4766138 rs11062709 |
| | | | | rs11062697 rs4766139 rs17769811 |
| | | | | rs17769793 rs17696868 rs3741936 |
| | | | | rs11062710 rs7962508 rs6489479 |
| | | | | rs12833949 rs917600 rs10848884 |
| | | | | rs12299470 rs17769918 rs2159347 |
| | | | | rs17769657 rs11062725 rs17769699 |
| | | | | rs17769758 |
| HSD3B2 | 0.7348 | 0.9966 | 6 | rs1819698 rs4659175 rs12141041 |
| | | | | rs1341018 rs17023577 rs1856886 |
| UGT1A4 | 0.7441 | 0.9966 | 27 | rs4124874 rs4399719 rs12052787 |
| | | | | rs3732220 rs3806595 rs4663945 |
| | | | | rs17862875 rs17864701 rs6714634 |
| | | | | rs11891311 rs1875263 rs11568318 |
| | | | | rs1042640 rs11563250 rs11563251 |
| | | | | rs929596 rs2003569 rs4148328 |
| | | | | rs4148329 rs6719561 rs28946889 |
| | | | | rs8330 rs10929303 rs2011404 |
| | | | | rs2302538 rs12468543 rs1018124 |
| ARSE | 0.7963 | 0.9966 | 4 | rs211641 rs211640 rs5982925 |
| | | | | rs211644 |
| UGT1A8 | 0.8013 | 0.9966 | 55 | rs3732220 rs3806595 rs4663945 |
| | | | | rs4124874 rs4399719 rs6736508 |
| | | | | rs6753320 rs12052787 rs6707947 |
| | | | | rs6760588 rs12474980 rs4485562 |
| | | | | rs6725478 rs17862875 rs17864701 |
| | | | | rs6714634 rs10929251 rs10929252 |
| | | | | rs17864689 rs7585521 rs11568318 |
| | | | | rs10168416 rs41564555 rs11673726 |
| | | | | rs2741045 rs1042640 rs11563250 |
| | | | | rs11563251 rs7572563 rs7592624 |
| | | | | rs1105880 rs929596 rs2003569 |
| | | | | rs11893247 rs17863784 rs2602381 |
| | | | | rs1823803 rs4148328 rs4148329 |
| | | | | rs7571337 rs6719561 rs6751673 |
| | | | | rs1113193 rs28946889 rs7608713 |
| | | | | rs8330 rs2602374 rs10929303 |
| | | | | rs2011404 rs4233633 rs2302538 |
| | | | | rs10176426 rs12468543 rs1018124 |
| | | | | rs28898590 |
| UGT1A5 | 0.8507 | 0.9966 | 30 | rs3732220 rs3806595 rs4663945 |
| | | | | rs4124874 rs4399719 rs12052787 |
| | | | | rs17862875 rs17864701 rs6714634 |
| | | | | rs11891311 rs1875263 rs11568318 |
| | | | | rs1042640 rs11563250 rs11563251 |
| | | | | rs929596 rs2003569 rs7572563 |
| | | | | rs4148328 rs4148329 rs6719561 |
| | | | | rs28946889 rs8330 rs10929303 |
| | | | | rs2011404 rs4233633 rs2302538 |
| | | | | rs12468543 rs1018124 rs28898590 |
| UGT1A10 | 0.8508 | 0.9966 | 55 | rs3732220 rs3806595 rs4663945 |
| | | | | rs4124874 rs4399719 rs6736508 |
| | | | | rs6753320 rs12052787 rs6707947 |
| | | | | rs12474980 rs4485562 rs6725478 |
| | | | | rs17862875 rs17864701 rs6714634 |
| | | | | rs10929251 rs10929252 rs6731242 |
| | | | | rs6760588 rs17864689 rs7585521 |
| | | | | rs11568318 rs10168416 rs11673726 |
| | | | | rs2741045 rs41564555 rs1042640 |
| | | | | rs11563250 rs11563251 rs7572563 |
| | | | | rs7592624 rs1105880 rs929596 |
| | | | | rs2003569 rs11893247 rs17863784 |
| | | | | rs2602381 rs4148328 rs4148329 |
| | | | | rs7571337 rs6719561 rs6751673 |
| | | | | rs1113193 rs28946889 rs7608713 |
| | | | | rs8330 rs2602374 rs10929303 |
| | | | | rs2011404 rs4233633 rs2302538 |
| | | | | rs10176426 rs12468543 rs1018124 |
| | | | | rs28898590 |
| ESR2 | 0.8523 | 0.9966 | 24 | rs960069 rs12435857 rs1256030 |
| | | | | rs1256114 rs1952585 rs8018687 |
| | | | | rs6573553 rs2978381 rs17179740 |
| | | | | rs928554 rs2772163 rs10137185 |
| | | | | rs7159462 rs1255998 rs1887994 |
| | | | | rs1048315 rs2357479 rs4986938 |
| | | | | rs17766755 rs8006145 rs3020443 |
| | | | | rs1256062 rs12434245 rs1256063 |
| LCMT2 | 0.8743 | 0.9966 | 4 | rs956391 rs514438 rs7048 rs3742970 |
| UGT1A9 | 0.8754 | 0.9966 | 46 | rs3732220 rs3806595 rs4663945 |
| | | | | rs4124874 rs4399719 rs6736508 |
| | | | | rs6753320 rs12052787 rs6707947 |
| | | | | rs12474980 rs4485562 rs6725478 |
| | | | | rs17862875 rs17864701 rs6714634 |
| | | | | rs11568318 rs10168416 rs11673726 |
| | | | | rs2741045 rs1042640 rs11563250 |
| | | | | rs11563251 rs17864689 rs7572563 |
| | | | | rs7592624 rs1105880 rs929596 |
| | | | | rs6731242 rs2003569 rs17863784 |
| | | | | rs2602381 rs4148328 rs4148329 |
| | | | | rs6719561 rs6751673 rs28946889 |
| | | | | rs13418420 rs8330 rs10929303 |
| | | | | rs2011404 rs4233633 rs2302538 |
| | | | | rs10176426 rs12468543 rs1018124 |
| | | | | rs28898590 |
| AR | 0.8859 | 0.9966 | 3 | rs4827547 rs7064188 rs2361634 |
| UGT1A6 | 0.8895 | 0.9966 | 41 | rs3732220 rs3806595 rs4663945 |
| | | | | rs4124874 rs4399719 rs12052787 |
| | | | | rs6736508 rs6753320 rs11891311 |
| | | | | rs1875263 rs17862875 rs17864701 |
| | | | | rs6714634 rs11568318 rs10168416 |
| | | | | rs12474980 rs4485562 rs6707947 |
| | | | | rs1042640 rs11563250 rs11563251 |
| | | | | rs7572563 rs7592624 rs1105880 |
| | | | | rs929596 rs2003569 rs17863784 |
| | | | | rs4148328 rs4148329 rs6719561 |
| | | | | rs6751673 rs10179094 rs28946889 |
| | | | | rs8330 rs10929303 rs2011404 |
| | | | | rs4233633 rs2302538 rs12468543 |
| | | | | rs1018124 rs28898590 |
| UGT1A7 | 0.8944 | 0.9966 | 41 | rs3732220 rs3806595 rs4663945 |
| | | | | rs4124874 rs4399719 rs6736508 |
| | | | | rs6753320 rs12052787 rs6725478 |
| | | | | rs6707947 rs12474980 rs4485562 |
| | | | | rs17862875 rs17864701 rs6714634 |
| | | | | rs11568318 rs10168416 rs11673726 |
| | | | | rs1042640 rs11563250 rs11563251 |
| | | | | rs7572563 rs7592624 rs1105880 |
| | | | | rs929596 rs2003569 rs17864689 |
| | | | | rs17863784 rs4148328 rs4148329 |
| | | | | rs6719561 rs6751673 rs28946889 |
| | | | | rs8330 rs10929303 rs2011404 |
| | | | | rs4233633 rs2302538 rs12468543 |
| | | | | rs1018124 rs28898590 |
| AKR1C4 | 0.91 | 0.9966 | 18 | rs17134533 rs7897431 rs1413781 |
| | | | | rs4880716 rs1334473 rs1931679 |
| | | | | rs11253048 rs9423382 rs11253042 |
| | | | | rs11253046 rs2151896 rs11253045 |
| | | | | rs12775790 rs7083869 rs7070862 |
| | | | | rs12247748 rs11594520 rs10904442 |
| STS | 0.9207 | 0.9966 | 11 | rs4132409 rs5978405 rs1131289 |
| | | | | rs6530079 rs5979315 rs5934770 |
| | | | | rs4403552 rs17268974 rs17268988 |
| | | | | rs5933863 rs12861247 |
| HSD17B8 | 0.9418 | 0.9966 | 4 | rs107822 rs110662 rs421446 |
| | | | | rs1547387 |
| ARSD | 0.9577 | 0.9966 | 3 | rs211653 rs5982925 rs6642067 |
| HSD17B2 | 0.9622 | 0.9966 | 15 | rs11642323 rs9934209 rs723012 |
| | | | | rs1364283 rs6564962 rs2966248 |
| | | | | rs11648233 rs8191248 rs3887358 |
| | | | | rs10514524 rs2042429 rs11860188 |
| | | | | rs996752 rs2955162 rs4291899 |
| HSD17B7 | 0.9888 | 0.9966 | 8 | rs4656381 rs2805053 rs2803865 |
| | | | | rs11589262 rs10917597 rs1780019 |
| | | | | rs1892125 rs1039874 |
| UGT1A1 | 0.9942 | 0.9966 | 19 | rs6714634 rs4124874 rs4399719 |
| | | | | rs11568318 rs1042640 rs11563250 |
| | | | | rs11563251 rs12052787 rs2003569 |
| | | | | rs4148328 rs4148329 rs6719561 |
| | | | | rs11673726 rs28946889 rs8330 |
| | | | | rs929596 rs10929303 rs2302538 |
| | | | | rs1018124 |
| UGT1A3 | 0.9966 | 0.9966 | 21 | rs4124874 rs4399719 rs12052787 |
| | | | | rs17862875 rs17864701 rs6714634 |
| | | | | rs11673726 rs11568318 rs1042640 |
| | | | | rs11563250 rs11563251 rs929596 |
| | | | | rs2003569 rs4148328 rs4148329 |
| | | | | rs6719561 rs28946889 rs8330 |
| | | | | rs10929303 rs2302538 rs1018124 |

Four SNPs (rs6900796, which flanks 3′ UTR; rs2326215 and rs6569442, which are in the coding region; and rs1268121, which is in an intron) in TMRT11 nucleic acid were further analyzed for time to progression on ADT. rs1268121 (A>G) exhibited an MAF of 15%; rs2326215 (A>G) exhibited an MAF of 37%; rs6569442 (A>C) exhibited an MAF of 33%; and rs6900796 (A>G) exhibited an MAF of 49%.

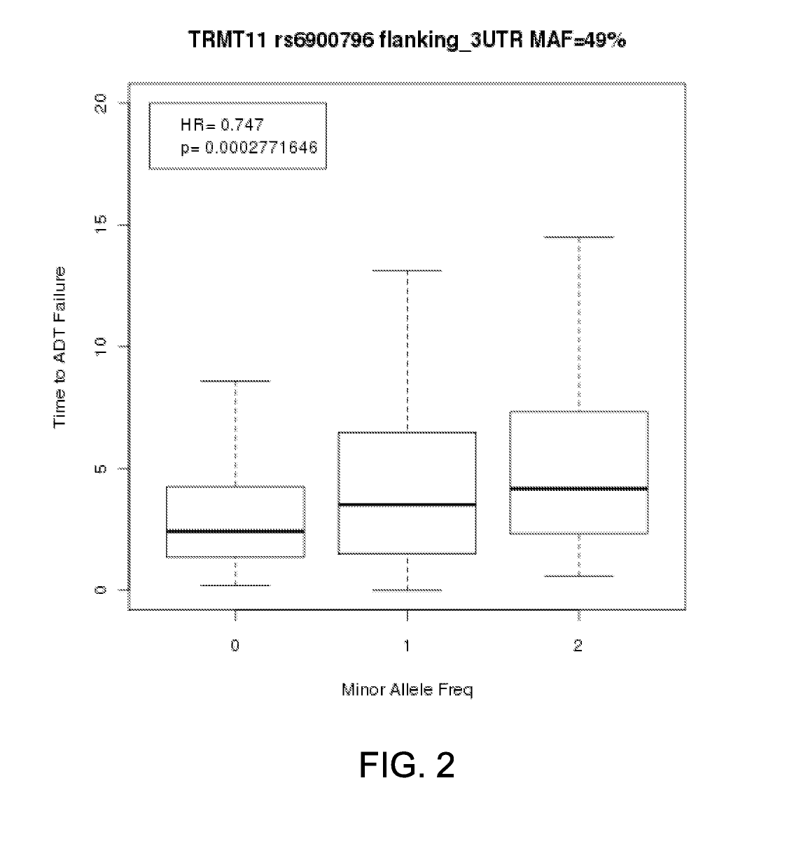

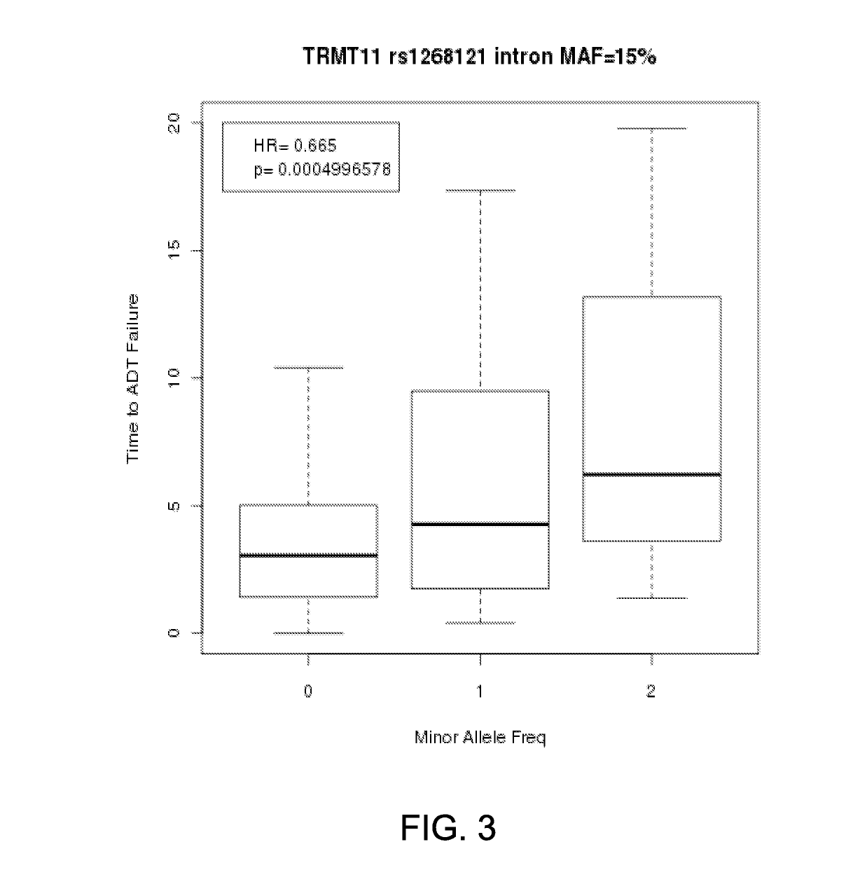

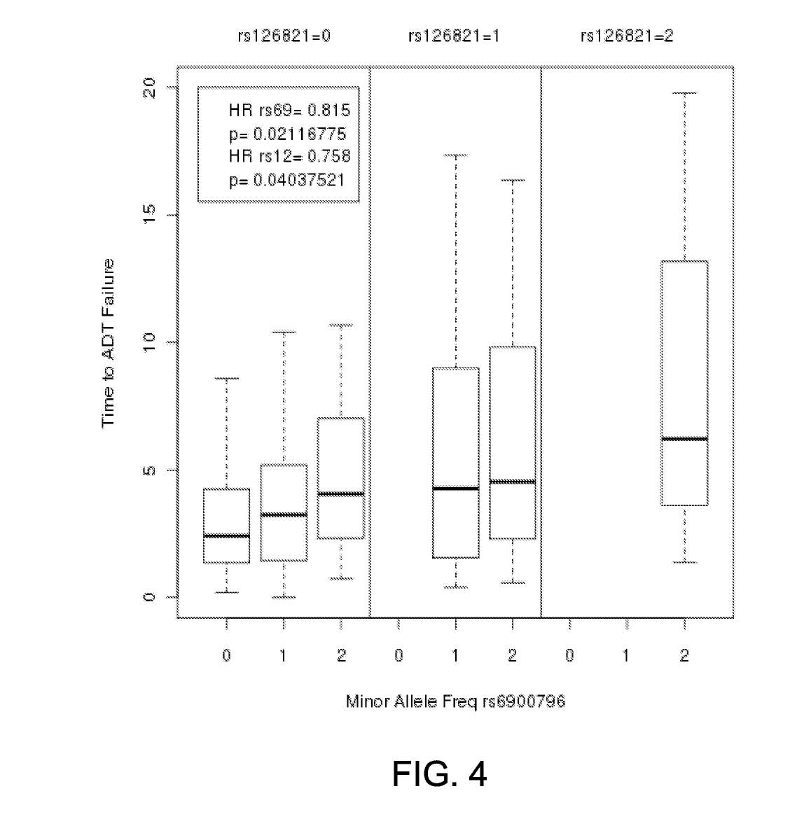

Of the four TRMT11 SNPs (rs1268121, rs2326215, rs6569442, and rs6900796) further analyzed for time to progression on ADT, two (rs1268121 and rs6900796) were found to be highly significant for duration of response to ADT. An overall protective effect was observed in the presence of 1 or 2 alleles for these SNPs (Table 4 and FIGS. 2-4).

TABLE 4

| | Minor Allele = 0 | Minor Allele = 1 | Minor Allele = 2 | p-value |
|---|---|---|---|---|
| TRMT11 | Median time to ADT | Median time to ADT | Median time to ADT | (Spearman |
| SNP | Failure | Failure | Failure | correlation co- |
| marker | (IQ: 25-75) (years) | (IQ: 25-75) (Years) | (IQ: 25-75) (Years) | efficient) |
| rs1268121 | 3.05 (1.42-5.02) | 4.27 (1.75-9.49) | 6.22 (3.62-13.17) | 0.002 (0.18) |
| rs2326215 | 3.01 (1.41-5.87) | 3.56 (1.64-6.29) | 3.85 (2.26-7.02) | 0.09 (0.09) |
| rs6569442 | 3.01 (1.41-5.87) | 3.56 (1.64-6.29) | 3.85 (2.26-7.02) | 0.09 (0.09) |
| rs6900796 | 2.42 (1.36-4.26) | 3.52 (1.51-6.48) | 4.18 (2.33-7.33) | 0.3 × 10⁻³ (0.20) |

Among the non-tagged candidate SNPs, four showed a significant association (p<0.05) with ADT response (Table 5). Two of the SNPs (rs10478424, rs11749784) were from HSD17B4 while the other SNPs were from CYP19A1 (rs2124872) and SREBF2 (rs11702960). However, none of these associations were confirmed with a FDR<0.10.

TABLE 4

| | | Principal | Principal | False |
|---|---|---|---|---|
| | | Components | Components | Discovery |
| | GENE | Analysis | Analysis Gleason | Rate |
| rsID | Symbol | P-value | Adjusted P-value | (q-value) |
| rs10478424 | HSD17B4 | 0.02 | 0.02 | 0.83 |
| rs2124872 | CYP19A1 | 0.04 | 0.31 | 0.83 |
| rs11702960 | SREBF2 | 0.04 | 0.22 | 0.83 |
| rs11749784 | HSD17B4 | 0.05 | 0.06 | 0.83 |

These results demonstrate that knowledge about the presence of variation in these hormone metabolizing genes can be used to predict the efficacy of ADT in individuals.

### Example 2

### Identifying Genotypic Markers Associated with Prostate Cancer Survival

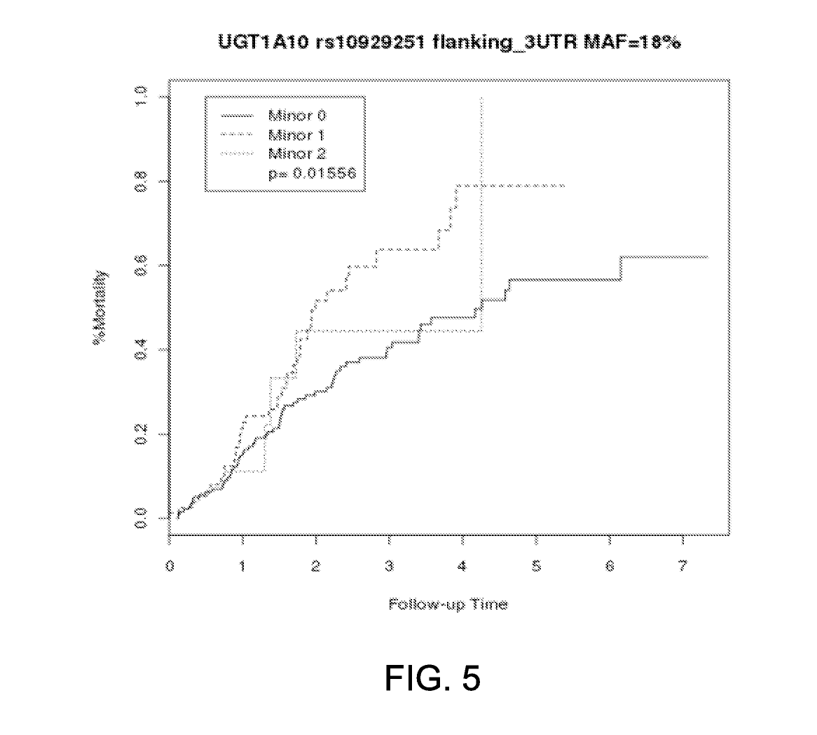

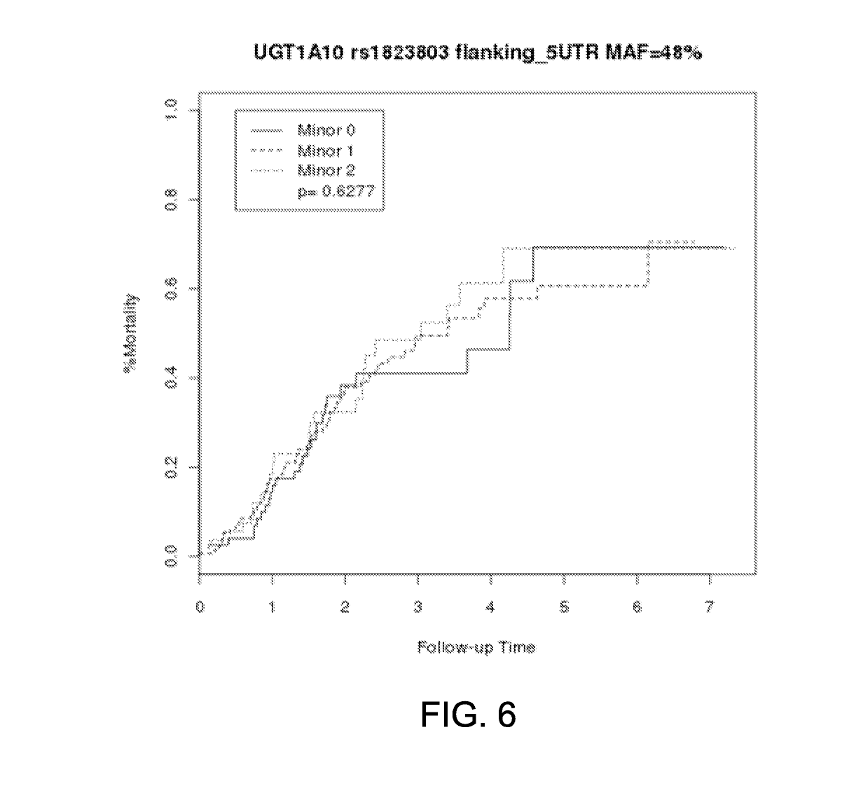

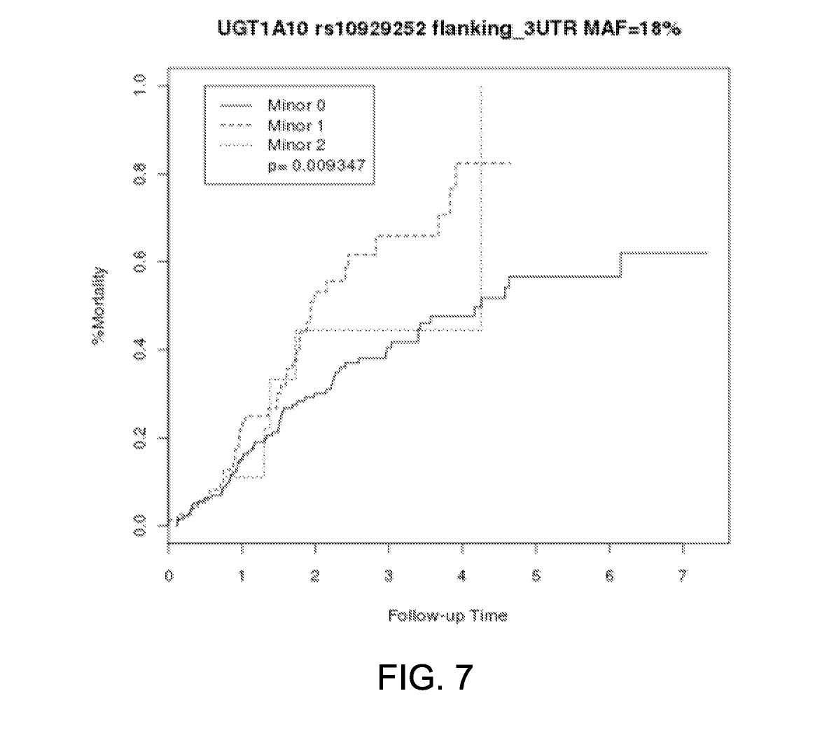

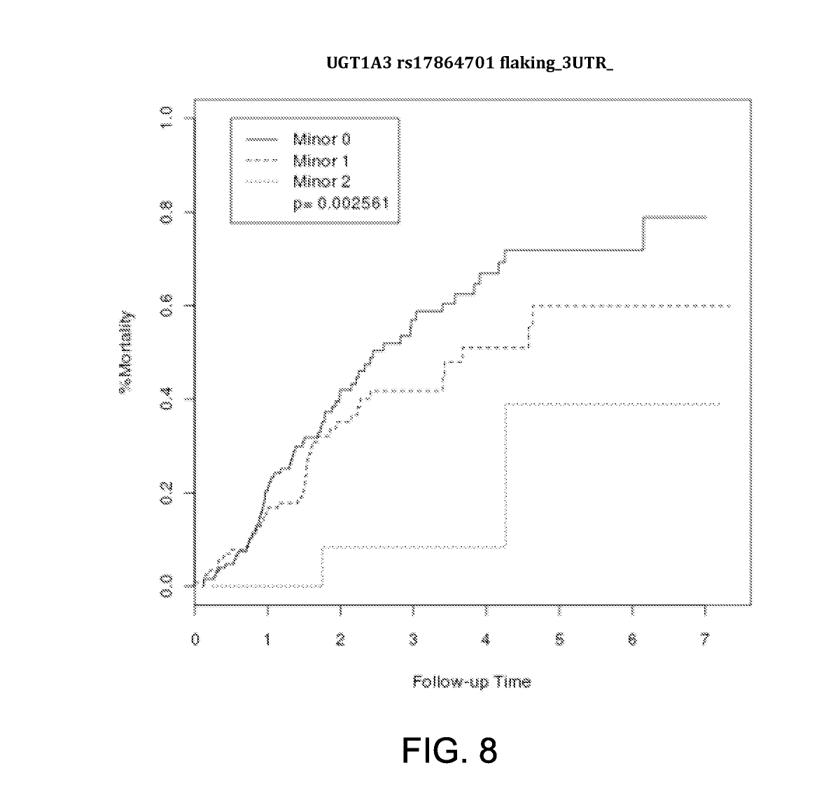

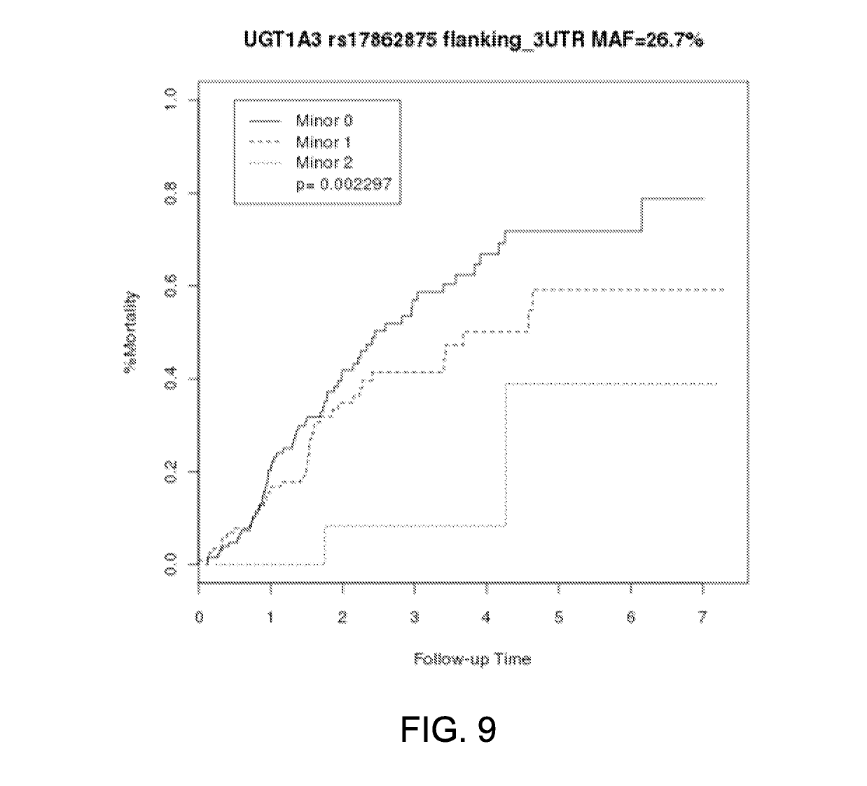

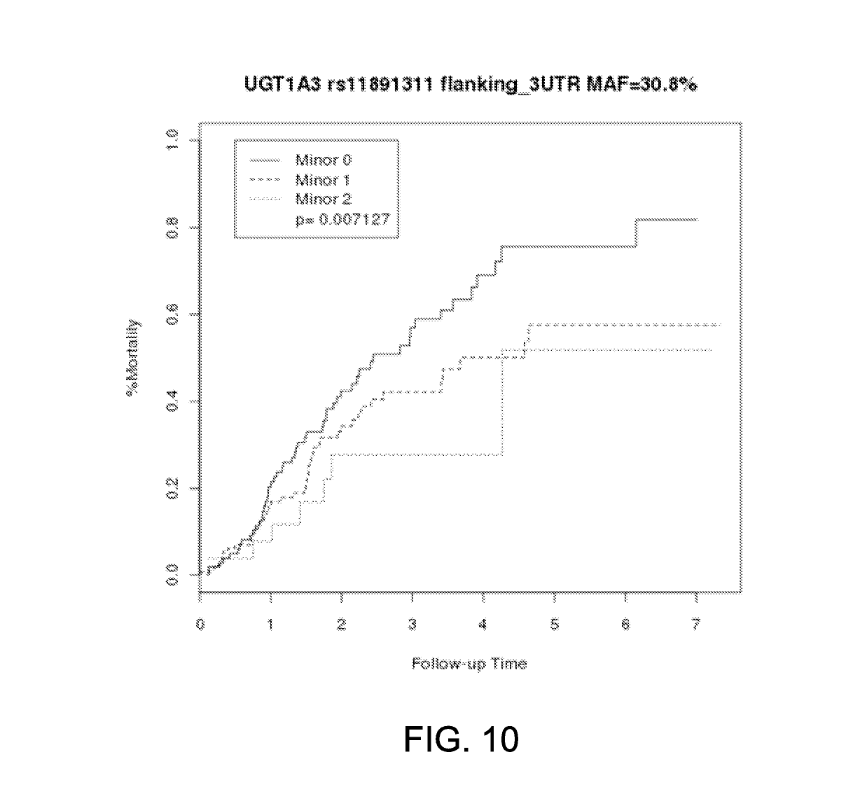

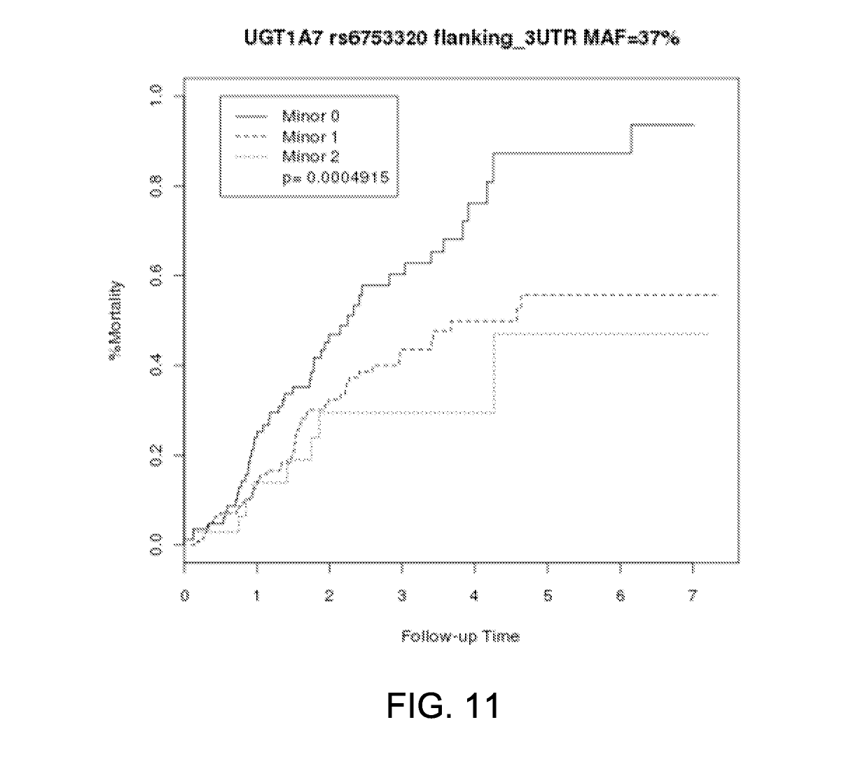

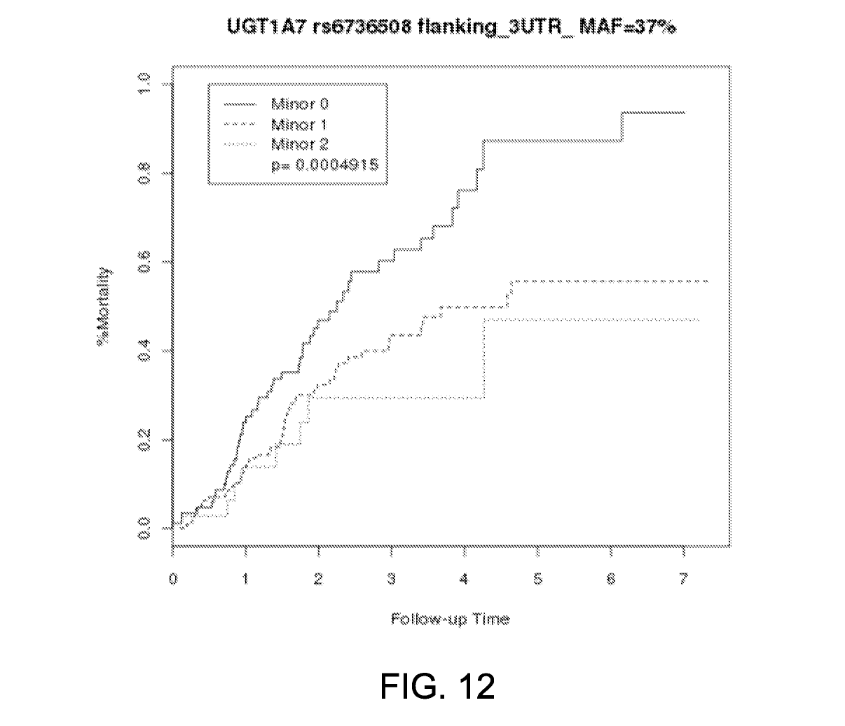

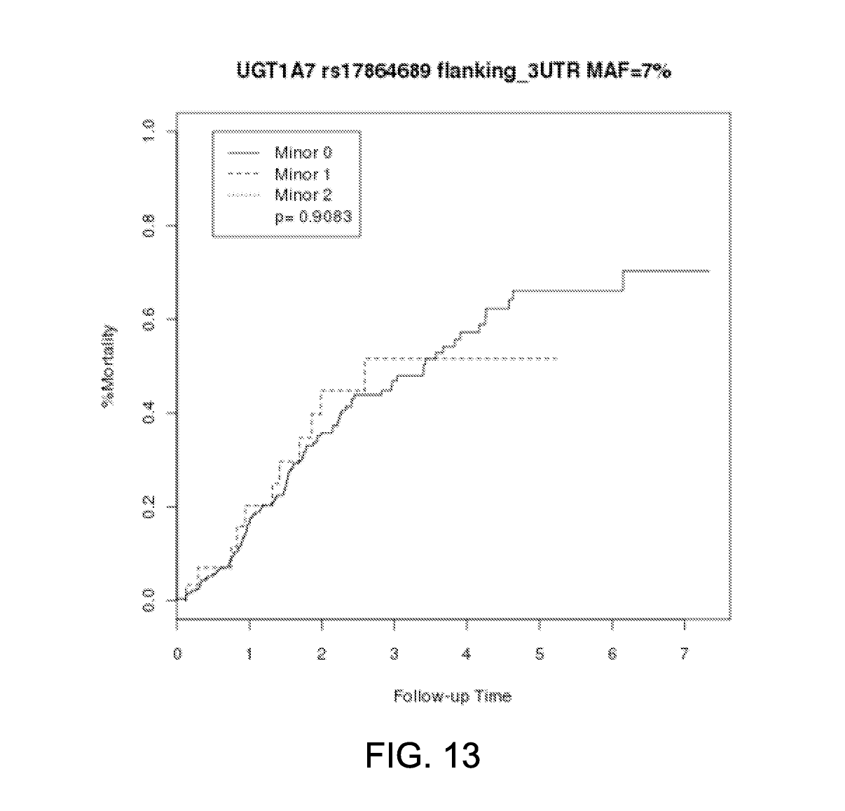

SNPs within UGT1A10, UGT1A7, and UGT1A3 nucleic acid were identified as being genetic markers capable of differentiating between prostate cancer patients likely to survive prostate cancer related death for a short period from those likely to survive prostate cancer related death for a long period, regardless of prostate cancer treatment (Table 2 and FIGS. 5-13). The summaries for UGT1A10, UGT1A7, and UGT1A3 in relation to prostate specific mortality for the 267 subjects are provided in Table 2. For UGT1A10, UGT1A7, and UGT1A3, the unadjusted association with mortality was significant with p-values=0.0059, 0.001745, and 0.003718, respectively, with corresponding FDR rates of 0.1918, 0.1710, and 0.1822. When adjusting for age, gleason score, and duration of ADT failure, the adjusted p-values were p=0.0052, 0.0062, and 0.0194, respectively, with corresponding FDR rates of 0.2922, 0.2922, and 0.61405.

TABLE 2
Mortality percentages.

| | | Follow-up Time in Years | | | | |
|---|---|---|---|---|---|---|
| Mortality | Minor | 1 | 2 | 3 | 4 | 5 |
| Gene-SNP | Allele | (percent) | (percent) | (percent) | (percent) | (percent) |
| UGT1A10rs10929251 | 0 | 15 | 30 | 41 | 48 | 57 |
| UGT1A10rs10929251 | 1 | 21 | 52 | 64 | 79 | 79 |
| UGT1A10rs10929251 | 2 | 11 | 44 | 44 | 44 | |
| UGT1A10rs10929252 | 0 | 15 | 30 | 41 | 48 | 57 |
| UGT1A10rs10929252 | 1 | 22 | 53 | 66 | 82 | |
| UGT1A10rs10929252 | 2 | 11 | 44 | 44 | 44 | |
| UGT1A10rs1823803 | 0 | 14 | 38 | 41 | 46 | 69 |
| UGT1A10rs1823803 | 1 | 17 | 38 | 50 | 58 | 61 |
| UGT1A10rs1823803 | 2 | 19 | 32 | 48 | 61 | 69 |
| UGT1A3rs11891311 | 0 | 20 | 42 | 57 | 69 | 76 |
| UGT1A3rs11891311 | 1 | 16 | 34 | 42 | 50 | 57 |
| UGT1A3rs11891311 | 2 | 7 | 28 | 28 | 28 | 52 |
| UGT1A3rs17862875 | 0 | 20 | 42 | 57 | 67 | 72 |
| UGT1A3rs17862875 | 1 | 16 | 35 | 41 | 50 | 59 |
| UGT1A3rs17862875 | 2 | 0 | 8 | 8 | 8 | 39 |
| UGT1A3rs17864701 | 0 | 20 | 42 | 57 | 67 | 72 |
| UGT1A3rs17864701 | 1 | 16 | 35 | 42 | 51 | 60 |
| UGT1A3rs17864701 | 2 | 0 | 8 | 8 | 8 | 39 |
| UGT1A7rs17864689 | 0 | 16 | 36 | 47 | 57 | 66 |
| UGT1A7rs17864689 | 1 | 20 | 45 | 52 | 52 | 52 |
| UGT1A7rs6736508 | 0 | 24 | 47 | 60 | 76 | 87 |
| UGT1A7rs6736508 | 1 | 13 | 32 | 44 | 50 | 56 |
| UGT1A7rs6736508 | 2 | 14 | 29 | 29 | 29 | 47 |
| UGT1A7rs6753320 | 0 | 24 | 47 | 60 | 76 | 87 |
| UGT1A7rs6753320 | 1 | 13 | 32 | 44 | 50 | 56 |
| UGT1A7rs6753320 | 2 | 14 | 29 | 29 | 29 | 47 |

These results demonstrate that information about these variations of these SNPs belonging to these genes in individual patients will allow to prognosticate patient survival in the advanced stage of prostate cancer.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for identifying a prostate cancer patient likely to respond to androgen deprivation therapy, wherein said method comprises:
(a) detecting the presence of a TMRT11 allele comprising rs6900796 or rs1268121 in said patient, and
(b) classifying said patient as being likely to respond to said androgen deprivation therapy without failure for a time greater than 3.5 years based at least in part on the presence of said TMRT11 allele.

2. The method of claim 1, wherein said prostate cancer patient is a human.

3. The method of claim 1, wherein said method comprises detecting the presence of a TMRT11 allele comprising rs6900796.

4. The method of claim 1, wherein said method comprises detecting the presence of a TMRT11 allele comprising rs1268121.

5. A method for identifying a prostate cancer patient likely to survive death related to prostate cancer for a time longer than 3.5 years, wherein said method comprises:
(a) detecting the presence of a UGT1A3 allele comprising rs17864701, rs17862875, or rs11891311 in said patient, and
(b) classifying said patient as being likely to survive death related to prostate cancer for a time longer than 3.5 years based at least in part on the presence of said UGT1A3 allele.

6. The method of claim 5, wherein said prostate cancer patient is a human.

7. The method of claim 5, wherein said method comprises detecting the presence of a UGT1A3 allele comprising rs17864701.

8. The method of claim 5, wherein said method comprises detecting the presence of a UGT1A3 allele comprising rs17862875.

9. The method of claim 5, wherein said method comprises detecting the presence of a UGT1A3 allele comprising rs11891311.

10. A method for identifying a prostate cancer patient likely to survive death related to prostate cancer for a time longer than 3.5 years, wherein said method comprises:
(a) detecting the presence of a UGT1A7 allele comprising rs6753320 or rs6736508 in said patient, and
(b) classifying said patient as being likely to survive death related to prostate cancer a time longer than 3.5 years based at least in part on the presence of said UGT1A7 allele.

11. The method of claim 9, wherein said prostate cancer patient is a human.

12. The method of claim 9, wherein said method comprises detecting the presence of a UGT1A7 allele comprising rs6753320.

13. The method of claim 9, wherein said method comprises detecting the presence of a UGT1A7 allele comprising rs6736508.

14. A method for identifying a prostate cancer patient likely to survive death related to prostate cancer for a time longer than 3.5 years, wherein said method comprises:
(a) detecting the presence of two UGT1A10 alleles comprising a wild-type sequence at the rs10929251 or rs10929252 SNP position in said patient, and
(b) classifying said patient as being likely to survive death related to prostate cancer a time longer than 3.5 years based at least in part on the presence of said two UGT1A10 alleles.

15. The method of claim 14, wherein said prostate cancer patient is a human.

16. The method of claim 14, wherein said method comprises detecting the presence of two UGT1A10 alleles comprising a wild-type sequence at the rs10929251 SNP position.

17. The method of claim 14, wherein said method comprises detecting the presence of two UGT1A10 alleles comprising a wild-type sequence at the rs10929252 SNP position.

18. A method for identifying a prostate cancer patient likely to survive death related to prostate cancer for a time shorter than 3.0 years, wherein said method comprises:
(a) detecting the presence of a UGT1A10 allele comprising rs10929251 or rs10929252 in said patient, and
(b) classifying said patient as being likely to survive death related to prostate cancer for a time shorter than 3.0 years based at least in part on the presence of said UGT1A10 allele.

19. The method of claim 18, wherein said prostate cancer patient is a human.

20. The method of claim 18, wherein said method comprises detecting the presence of a UGT1A10 allele comprising rs10929251.

21. The method of claim 18, wherein said method comprises detecting the presence of a UGT1A10 allele comprising rs10929252.
